(19) United States
(12) Patent Application Publication
(10) Pub. No.: US 2016/0158510 A1
(43) Pub. Date: Jun. 9, 2016

(54) CUTANEOUS TREATMENT APPARATUS AND METHODS OF USE

(71) Applicant: L'Oreal, Paris (FR)

(72) Inventors: Vincenzo Casasanta, III, Woodinville, WA (US); Chad Jason MacDonald, Bothell, WA (US)

(73) Assignee: L'OREAL, Paris (FR)

(21) Appl. No.: 14/563,863

(22) Filed: Dec. 8, 2014

(51) Int. Cl. A61M 37/00; A61M 35/00

(52) U.S. Cl. CPC A61M 37/00; A61M 35/00; A61M 2037/0007

(57) ABSTRACT

Cutaneous treatment apparatuses are provided that enhance percutaneous absorption of a treatment formulation. Related methods are also provided. In general, examples of the cutaneous treatment systems and/or apparatuses described herein are capable of attaching to a cutaneous surface and applying a treatment formulation to a localized cutaneous region. Embodiments of a cutaneous treatment apparatus generally include a heater and a treatment delivery component, including a treatment portion. The treatment portion includes a treatment formulation, including a therapeutic compound, and a treatment surface configured to contact the cutaneous region. The heater component is removably couplable with the treatment delivery component and is configured to transfer heat to the treatment portion. Upon heating, the therapeutic compound is configured to release from the treatment portion through the treatment surface towards the cutaneous region.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with one embodiment, a cutaneous treatment apparatus is provided. The cutaneous treatment apparatus generally includes a heater component including a heater, where the heater component has a heater component footprint defined by a heater component perimeter; and a first treatment delivery component including a treatment portion, where the treatment portion has a treatment portion footprint defined by a treatment portion perimeter. The treatment portion includes a treatment formulation, including a therapeutic compound, and a treatment surface configured to contact a cutaneous region. The heater component is removably couplable with the first treatment delivery component and is configured to transfer heat to the treatment portion, wherein the therapeutic compound is configured to release from the treatment portion through the treatment surface towards the cutaneous region upon heating.

In accordance with another embodiment, a method of applying a treatment to a cutaneous region, using a cutaneous treatment apparatus of any of the embodiments described herein, is provided. The method generally includes contacting the treatment surface to a portion of the cutaneous region and heating the treatment portion to a treatment temperature with the heater, thereby releasing the therapeutic compound through the treatment surface towards the cutaneous region.

In accordance with any of the embodiments described herein, the first treatment delivery component may further include a thermal distribution portion intermediate the treatment portion and the heater component; wherein the thermal distribution portion may be configured to distribute the heat to areas of the treatment portion outside the heater component footprint.

In accordance with any of the embodiments described herein, the heater component may be further configured to transfer heat through the treatment surface towards the cutaneous region.

In accordance with any of the embodiments described herein, the first treatment delivery component may further include an adhesive portion for removably attaching the treatment delivery component to the cutaneous region, such that the treatment surface contacts the cutaneous region.

In accordance with any of the embodiments described herein, the adhesive portion may expand beyond the treatment portion footprint to allow for contact with the cutaneous region.

In accordance with any of the embodiments described herein, the treatment portion footprint may be at least 10% larger than the heater component footprint.

In accordance with any of the embodiments described herein, the treatment portion footprint may be smaller than the heater component footprint.

In accordance with any of the embodiments described herein, the treatment formulation may be a hydrogel.

In accordance with any of the embodiments described herein, the therapeutic compound may be a cosmetic composition, including one or more moisturizers, humectants, whiteners, brighteners, epidermal fillers, peptides, saccharides, or acids.

In accordance with any of the embodiments described herein, the therapeutic compound may be a drug selected from the group consisting of: hormone therapies, pain management, and smoking cessation.

In accordance with any of the embodiments described herein, the heater component may be configured to removably couple with a second treatment delivery component after removal from the first treatment delivery component.

In accordance with any of the embodiments described herein, the treatment delivery component may further include a barrier intermediate the treatment portion and the heater component, wherein the barrier may prevent diffusion of the therapeutic compound away from the treatment surface.

In accordance with any of the embodiments described herein, the heater may be an electric heater.

In accordance with any of the embodiments described herein, the electric heater may include a ceramic insulator.

In accordance with any of the embodiments described herein, the heater component may include a controller configured to control a temperature and a target duration of heat production by the heater.

In accordance with any of the embodiments described herein, the controller may be configured to shut off power to the heater after the target duration of heat production has ended.

In accordance with any of the embodiments described herein, the heater component may be removably couplable by an attachment mechanism selected from the group consisting of mechanical, magnetic, and adhesive.

In accordance with any of the embodiments described herein, the heater may be powered by a battery.

In accordance with any of the embodiments described herein, the battery may be rechargeable.

In accordance with any of the embodiments described herein, the method of heating the treatment portion to the treatment temperature with the heater may include heating the treatment portion between about 35° and 45° Celsius.

In accordance with any of the embodiments described herein, the method may further include removably attaching a treatment delivery component to a cutaneous region with an adhesive portion.

In accordance with any of the embodiments described herein, the method of heating the treatment portion to the treatment temperature with the heater may include heating a portion of the cutaneous region.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a perspective view of two cutaneous treatment apparatuses, in accordance with the disclosed embodiments, showing the two cutaneous treatment apparatuses coupled to cutaneous regions of a user's face;

FIG. 2 is a perspective view of one cutaneous treatment apparatus of FIG. 1, with the heater component separated from a treatment delivery package;

FIG. 3 is a side view of one cutaneous treatment apparatus of FIG. 1, with the heater component separated from the treatment delivery package, and the protective covers of the treatment delivery package being peeled away, exposing the treatment delivery component;

FIG. 4 is a perspective view of one cutaneous treatment apparatus of FIG. 1, with the heater component removably coupled to the treatment delivery component;

FIG. 5 is a top view of one cutaneous treatment apparatus of FIG. 1, with the heater component removably coupled to the treatment delivery component;

FIG. 6 is a side view of one cutaneous treatment apparatus of FIG. 1, with the heater component removably coupled to the treatment delivery component;

FIG. 7 is a front view of one cutaneous treatment apparatus of FIG. 1, with the heater component removably coupled to the treatment delivery component;

FIG. 8 is an exploded view of a heater component of one cutaneous treatment apparatus of FIG. 1;

FIG. 9 is an exploded view of a treatment delivery package of one cutaneous treatment apparatus of FIG. 1, showing the treatment delivery component contained therein;

FIG. 10 is a bottom perspective view of a treatment portion of the treatment delivery component of one cutaneous treatment apparatus of FIG. 1, showing a treatment formulation contained therein;

FIGS. 11A and 11B are side views of a cutaneous treatment apparatus in accordance with the disclosed embodiments, showing the snap and catch removable coupling mechanism;

FIG. 12A is a bottom view of a heater component of a cutaneous treatment apparatus in accordance with the disclosed embodiments, showing slot linear rails of a slot removable coupling mechanism;

FIGS. 12B and 12C are side views of the cutaneous treatment apparatus of FIG. 12A, showing the slot removable coupling mechanism;

FIG. 13A is a bottom view of a heater component of a cutaneous treatment apparatus in accordance with the disclosed embodiments, showing pin attachment posts of a pin removable coupling mechanism;

FIGS. 13B and 13C are side views of the cutaneous treatment apparatus of FIG. 13A, showing the pin removable coupling mechanism;

FIG. 14 is a side view of a cutaneous treatment apparatus in accordance with the disclosed embodiments, showing a magnetic removable coupling mechanism;

FIG. 15 is a side view of a cutaneous treatment apparatus in accordance with the disclosed embodiments, showing a threaded ring removable coupling mechanism; and

FIG. 16 is a side view of a cutaneous treatment apparatus in accordance with the disclosed embodiments, showing an adhesive removable coupling mechanism.

### DETAILED DESCRIPTION

The detailed description set forth below in connection with the appended drawings, where like numerals reference like elements, is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein are interchangeable with other steps, or combinations of steps, in order to achieve the same or substantially similar result.

In the following description, numerous specific details are set forth in order to provide a thorough understanding of exemplary embodiments of the present disclosure. It will be apparent to one skilled in the art, however, that many embodiments of the present disclosure may be practiced without some or all of the specific details. In some instances, well-known process steps have not been described in detail in order not to unnecessarily obscure various aspects of the present disclosure. Further, it will be appreciated that embodiments of the present disclosure may employ any combination of features described herein.

Topical application of active treatment on skin is used for a wide variety of therapies. Some examples include: skin whitening, wrinkle reduction, skin toning, burn soothing, age spot removal, psoriasis control, vitamin infusion, eczema treatment, general moisturizing, cellulite smoothing, rash reduction, and skin tightening. Traditionally, the treatment of localized cutaneous regions has involved the simple application of a treatment formulation to the cutaneous surface.

The effectiveness of topical treatment is affected by a number of factors. In general, a significant impediment is the low permeability of the cutaneous surface, resulting in difficulty of treatment diffusion to the cutaneous layers. The outer cutaneous layer, the stratum corneum, serves as a barrier to treatment particles. When it is desired for the treatment formulation to reach beyond the stratum corneum, increased permeability is beneficial to promote percutaneous absorption. Several methods of increasing cutaneous permeability exist, including various mechanical stressors like abrasion and exfoliation, which essentially damage the cutaneous surface. Another method of increasing permeability, ultraviolet radiation, causes restructuring of the surface cells and weakens the boundary between the stratum corneum and epidermal layer. Chemicals, such as certain types of acid, can also be applied to the cutaneous surface to increase permeability. Finally, electroportation, or the application of short pulses of electric fields, has been shown to increase permeability. Each of these methods can cause cutaneous damage, irritation, and discomfort.

Therefore, a need exists for a treatment delivery system that enhances the permeability of the stratum corneum, increasing the percutaneous absorption of a treatment formulation, without the need to damage the cutaneous surface, cause irritation and discomfort, or apply harsh chemicals.

Embodiments of the present disclosure are generally directed to cutaneous treatment systems with enhanced percutaneous absorption and/or apparatus for applying treatment to a cutaneous region. In general, examples of the cutaneous treatment system and/or apparatus described herein are capable of attaching to a cutaneous surface and applying a treatment formulation to a localized cutaneous region. The embodiments illustrated in the FIGURES have been designed for use with cosmetic treatment of a cutaneous region (e.g., moisturizers, humectants, whiteners, brighteners, epidermal fillers, peptides, saccharides, acids, etc.). However, embodiments of the present disclosure are also used for drug delivery during cutaneous treatment applications (e.g., hormone therapies, pain management, smoking cessation, etc.), and are also within the scope of this disclosure. Cosmetic and drug treatments used with the cutaneous treatment system may include different therapeutic compounds to achieve the desired resulting treatment. In some instances, the cosmetic and drug treatments are “heat activated,” such that the application of heat increases diffusion of the therapeutic compound from a hydrogel, cutaneous absorption of the therapeutic compound, and/or efficacy of the treatment.

Embodiments of the cutaneous treatment apparatus of the present disclosure generally include a heater component including a heater, where the heater component has a heater component footprint defined by a heater component perimeter; and a first treatment delivery component including a treatment portion, where the treatment portion has a treatment portion footprint defined by a treatment portion perimeter. The treatment portion includes a treatment formulation, including a therapeutic compound, and a treatment surface configured to contact a cutaneous region. The heater component is removably couplable with the first treatment delivery component and is configured to transfer heat to the treatment portion, wherein the therapeutic compound is configured to release from the treatment portion through the treatment surface towards the cutaneous region upon heating.

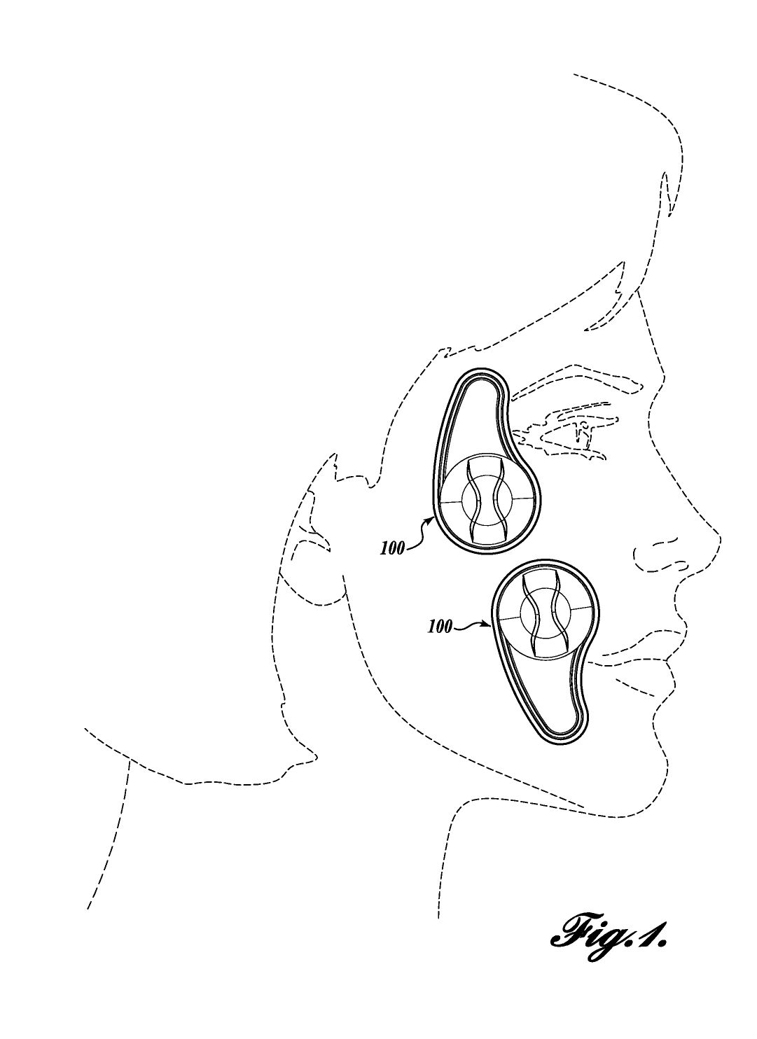

Referring to FIG. 1, a cutaneous treatment apparatus 100 in accordance with one embodiment of the present disclosure is provided. As an example, in FIG. 1, multiple cutaneous treatment apparatuses 100 are shown attached to various cutaneous regions on a user's face as examples of areas where treatment is desired. However, in other embodiments, a single cutaneous treatment apparatus 100 is used for treatment. Likewise, the cutaneous treatment apparatus 100 is attached to any suitable cutaneous region for a target duration, which constitutes a treatment session.

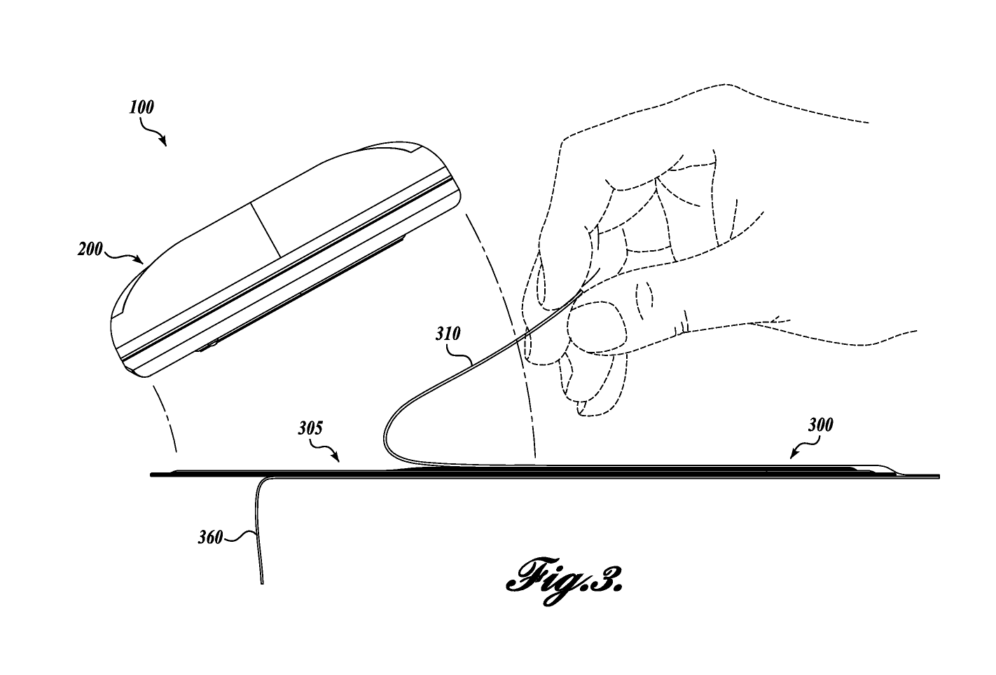

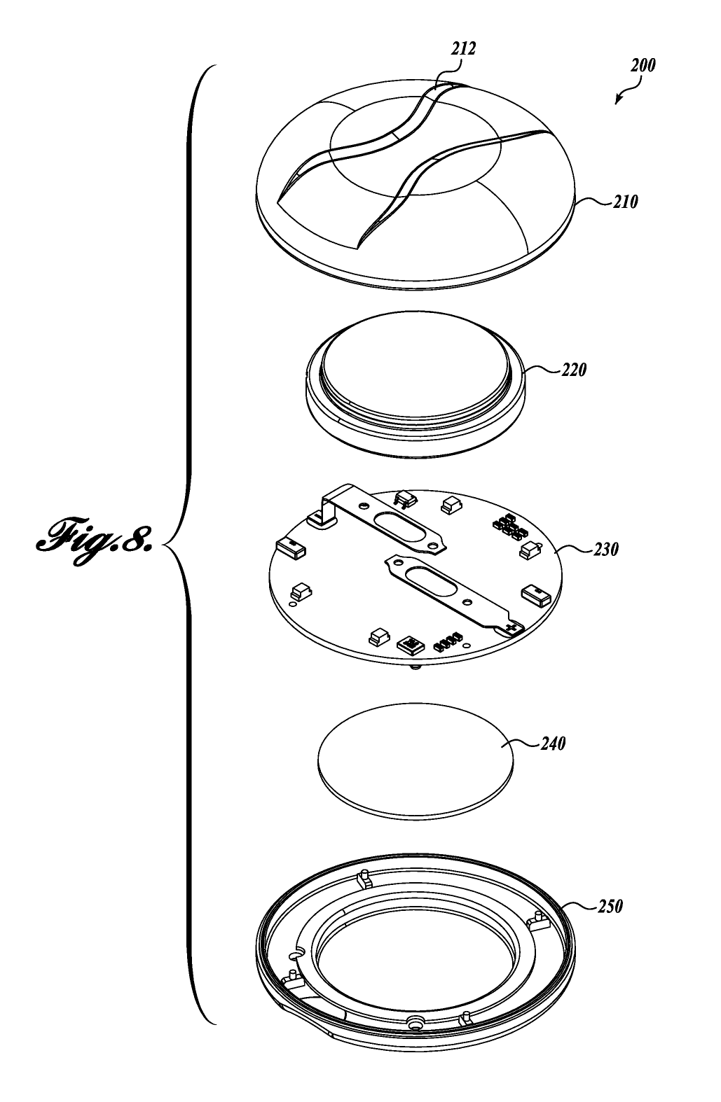

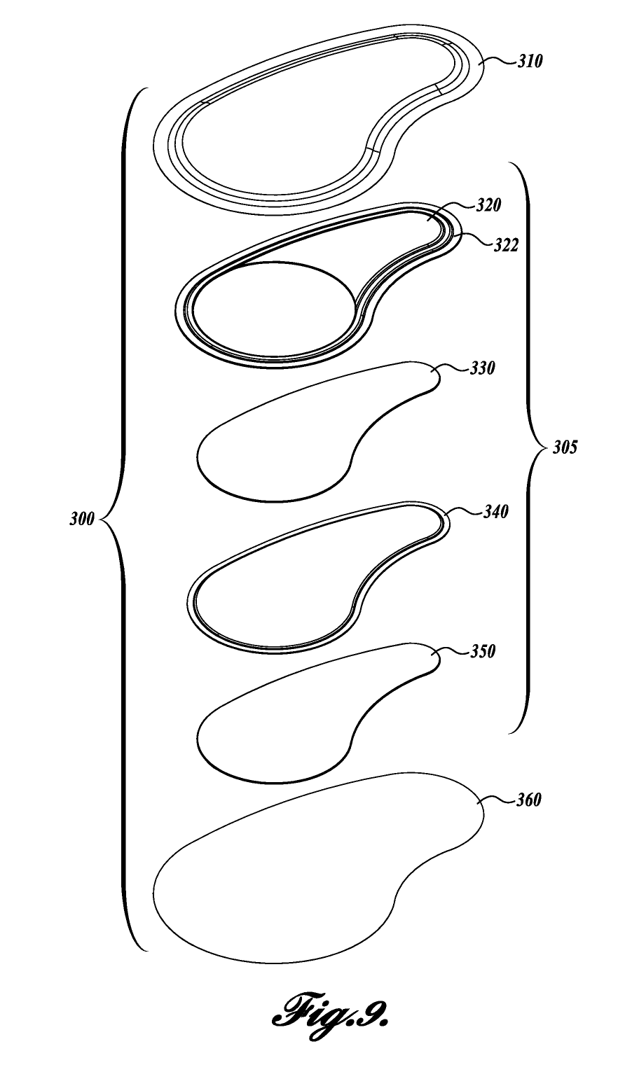

Referring now to FIG. 4, the cutaneous treatment apparatus 100 is shown with the heater component 200 removably coupled to the treatment delivery component 305. Various attachment mechanisms for removably coupling the heater component 200 are within the scope of the present disclosure. As non-limiting examples, the heater component 200 is removably couplable using mechanical, magnetic, or adhesive coupling (see, e.g., alternate embodiments in FIGS. 11-16). As explained in greater detail below, in some embodiments, the heater component 200 is intended to be reusable with additional treatment sessions, while the treatment delivery component 305 is only intended to be used for a single treatment session and then replaced. However, in other embodiments, the treatment delivery component 305 is reusable after application of additional treatment formulation 352 (see FIG. 10).

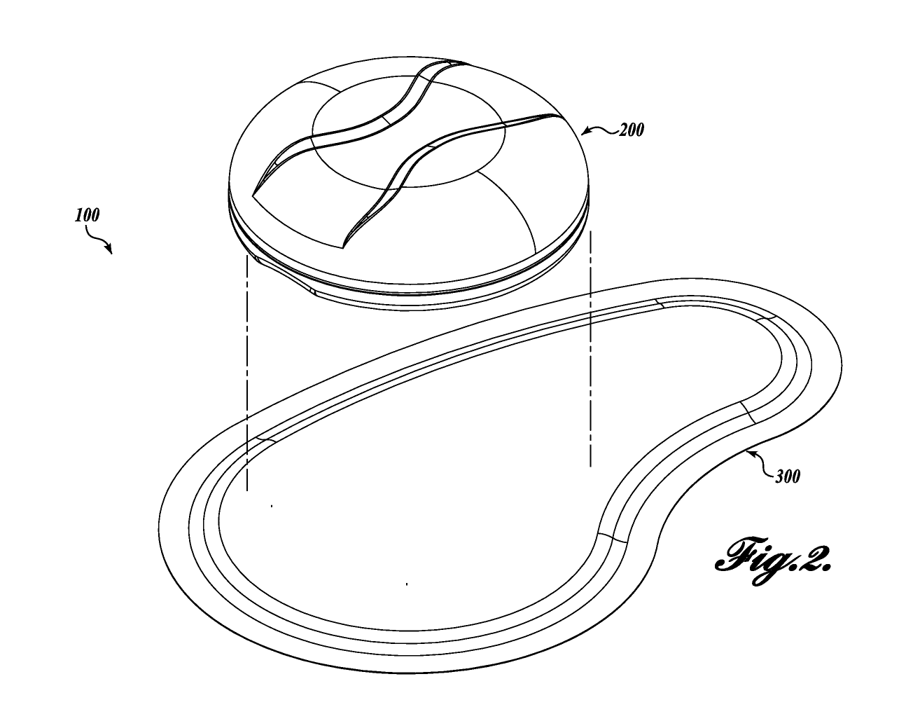

Referring to FIGS. 2 and 3, another embodiment of the cutaneous treatment apparatus 100 is shown. The cutaneous treatment apparatus 100 includes a heater component 200 that provides controlled heating, and a treatment delivery package 300 containing treatment delivery component 305, including the treatment formulation 352, for application to a cutaneous region. In the illustrated embodiment of FIGS. 2 and 3, the treatment delivery package 300 includes an upper coated storage protector 310 and a lower coated storage protector 360, which enclose the treatment delivery component 305, and are removed before use by peeling the upper coated storage protector 310 and the lower coated storage protector 360 away from the treatment delivery component 305, as shown in FIG. 3. After the upper coated storage protector 310 and the lower coated storage protector 360 are removed, the heater component 200 is removably coupled to the treatment delivery component 305, shown as coupled in FIG. 4, in preparation for applying the cutaneous treatment apparatus 100 to the cutaneous region. The storage protectors ensure sterility of the surfaces of the treatment delivery component 305 and prevent loss of the treatment formulation 352 from the treatment portion 350 (see FIG. 10). In some embodiments, the packaging of the treatment delivery component 305 serves the function of the storage protectors such that the storage protectors are omitted.

Orientation language, such as “top,” “bottom,” “upper,” “lower,” “under,” “below,” and “underside,” are used in the specification with reference to the orientation of the cutaneous treatment apparatus 100 as shown in FIG. 4. This language is intended to help the reader and is not intended to be limiting.

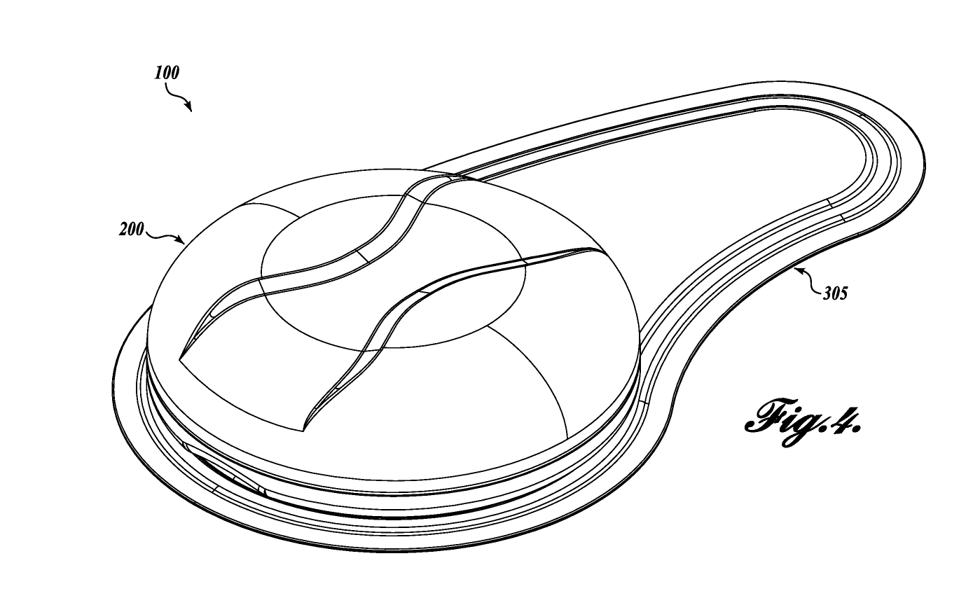

Referring now to FIG. 5, the top of an assembled cutaneous treatment apparatus 100 is shown. The heater component 200 of the illustrated embodiment is substantially cylindrical in shape. The outer circumference of the cylinder, when viewed from the top, defines a heater component perimeter 110. The area within the heater component perimeter 110, in the plane of the user's skin, defines a heater component footprint. Likewise, the outer edge of the treatment delivery component 305 defines a treatment delivery component perimeter 120. The area within the treatment delivery component perimeter 120, in the plane of the user's skin, defines a treatment delivery component footprint. In the illustrated embodiment, the heater component footprint is smaller than the treatment delivery component footprint. However, in other embodiments, the heater component footprint and the delivery component footprint are of equal size. In another embodiment, the heater component footprint is larger than the treatment delivery component footprint.

Further, in the illustrated embodiment, the heater component perimeter 110 is substantially circular in shape. However, in other embodiments, the heater component perimeter is produced in any number of shapes, including rectangular, oval, polygonal, or curvilinear. Likewise, in the illustrated embodiment, the treatment delivery component perimeter 120 is curvilinear in shape. However, in other embodiments, the treatment delivery component perimeter is produced in any number of shapes, including circular, rectangular, oval, polygonal, or variations of curvilinear. One advantage of the independent shape characteristics of each component is the ability to configure the shape of a treatment delivery component to conform to targeted cutaneous regions without the need to change the shape of the heater component for each of the areas to be treated.

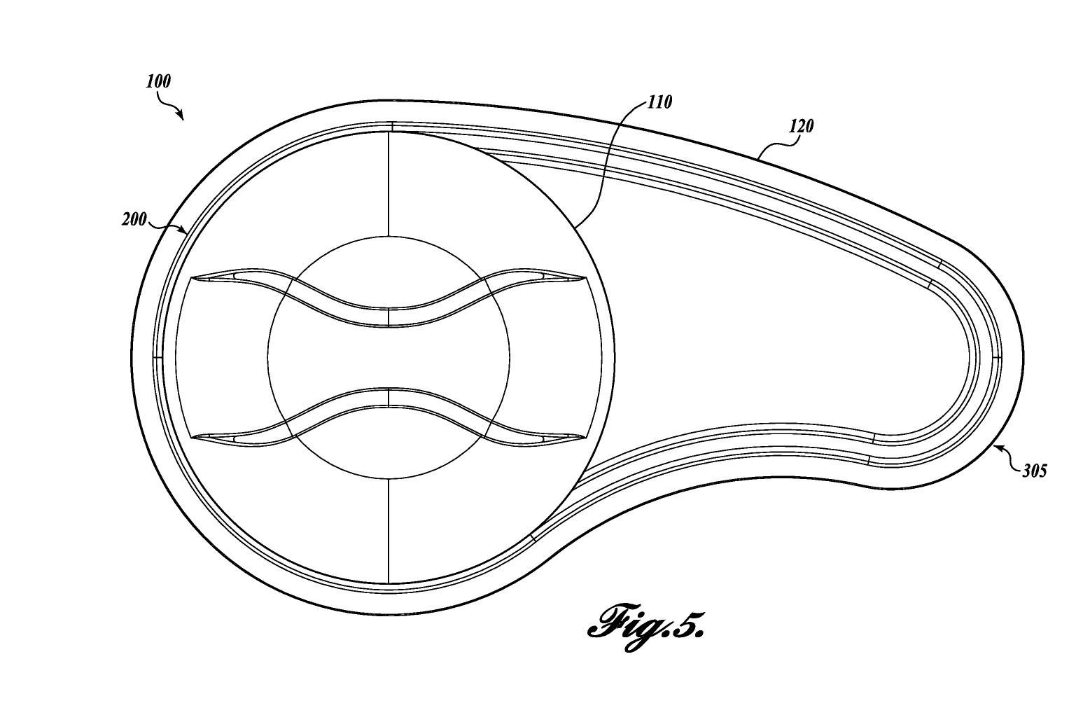

Referring now to FIGS. 6 and 7, the cutaneous treatment apparatus 100 of the present disclosure is shown in side and front elevation views. FIGS. 6 and 7 further show the interface of the heater component 200 with the treatment delivery component 305. More specifically, the heater component 200 further includes a heater top cover 210 and a heater bottom cover 250. The treatment delivery component 305 further includes a support layer 320. As illustrated, the heater bottom cover 250 and the support layer 320 interface to provide a removable coupling to prepare the cutaneous treatment apparatus 100 for a treatment session. For simplicity, FIGS. 2 and 3 depict an “adhesive” removable coupling of the heater component 200 to the treatment delivery component 305, as further illustrated in FIG. 16, and explained in greater detail below. Various other embodiments of the removable coupling are shown in FIGS. 11-15 and will also be explained in greater detail below.

Referring to FIG. 8, an exploded view of the heater component 200 is shown. The heater component 200 includes the heater top cover 210, a battery 220, a heater controller 230, a ceramic heater 240, and the heater bottom cover 250. The heater top cover 210 may also include a heater light pipe 212 for aesthetic effect while the heater component 200 is in use. In some embodiments, the heater light pipe 212 is tied to a treatment session to indicate when the session is finished (e.g., by blinking, pulsing, fading, or shutting off). Audible indications, such as a beep or alarm to alert a user to the end of a session, are also within the scope of this disclosure.

The battery 220 powers the heater component 200 to provide electricity for the ceramic heater 240. In some embodiments, the battery 220 is rechargeable to enable restoration the capacity of the battery 220 of the heater component 200 for use with further treatment sessions. Although a rechargeable battery provides the most convenience to the user, other sources of power are within the scope of this disclosure, including solar, disposable battery, or corded power for use with an A/C outlet.

The heater controller 230 is included in the heater component 200 to control the temperature output of the ceramic heater 240 while in use. In some embodiments, the heater controller 230 is configured to monitor the duration the heat is applied to the cutaneous region until the duration reaches a target, i.e., a treatment session. In some embodiments, the heater controller 230 automatically shuts off power to the ceramic heater 240 in the event the treatment session has ended, or if the user manually ends the treatment session before the end of the target duration is reached. The cutaneous region and the effectiveness of the treatment session are both sensitive to the treatment temperature of the heater component 200. Temperature sensitivity varies from user to user and is also dependent on the cutaneous region in which the treatment is applied. In highly sensitive areas, a lower temperature is necessary to ensure comfort of the user during the treatment session. In tougher or thicker cutaneous regions, a higher temperature is needed to promote infusion of the active treatment. In the system of the present disclosure, the heater component output temperature heats the cutaneous region between about 35° Celsius to 45° Celsius. In other embodiments, the output temperature of the heater component 200 of the cutaneous treatment apparatus 100 heats the cutaneous region between about 42° Celsius to 44° Celsius. In other embodiments, the output temperature of the heater component 200 of the cutaneous treatment apparatus 100 heats the cutaneous region less than about 45° Celsius. As used herein, the term “about” indicates +/−5%, unless noted otherwise.

Still referring to FIG. 8, a ceramic heater 240 is shown. The ceramic heater 240 uses power from the battery 220 to produce heat through resistive heating of wire elements (not shown) located within the ceramic heater 240. The advantage of using a ceramic heater is the ability to confine the heater to a small package, as well as the relative simplicity of the components when compared to other heating methods. Although a ceramic heater is used in the illustrated embodiment, other heating systems, such as radiant or forced convection heating, are also within the scope of this disclosure. Likewise, the ceramic heater 240 is illustrated in the present disclosure as having a substantially cylindrical shape; however, any suitable shape of ceramic heater is also within the scope of this disclosure.

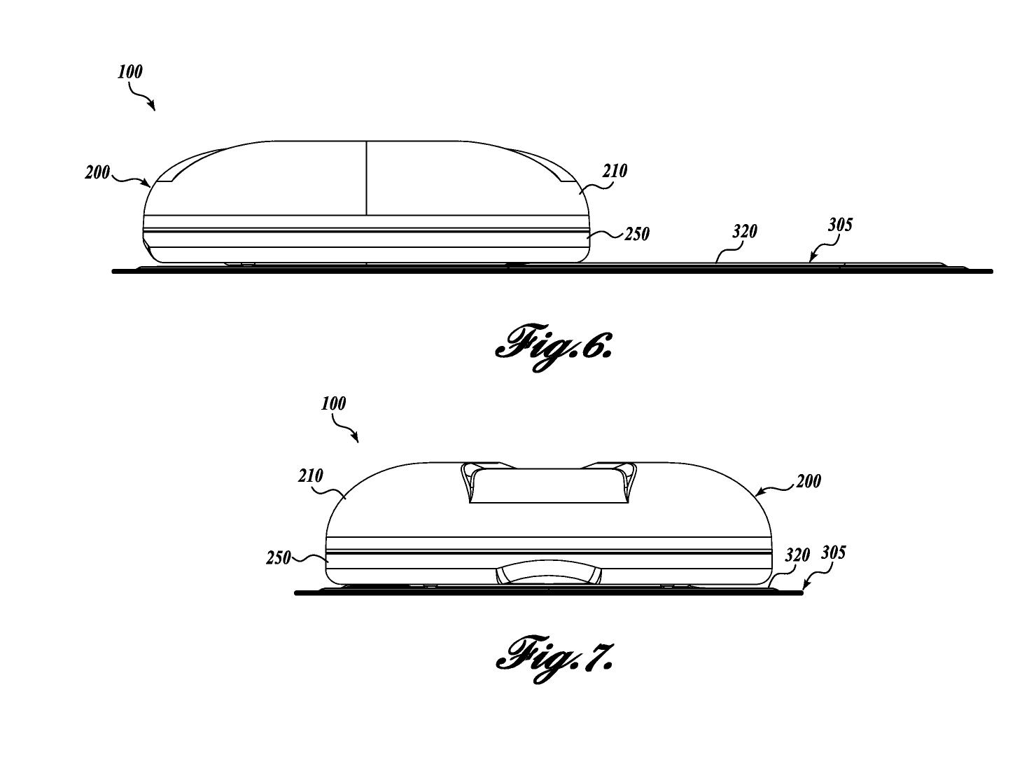

Referring now to FIG. 9, an exploded view of the treatment delivery component 305 is shown. The treatment delivery component 305 includes, a support layer 320, a thermal distribution layer 330, a barrier layer 340, and a treatment portion 350. In some embodiments, the upper coated storage layer 310 is applied to the treatment delivery component 305. Likewise, in other embodiments, the lower coated storage layer 360 is applied to the treatment delivery component 305. Embodiments that include assembly of both the upper coated storage layer 310 and the lower coated storage layer 360 to the treatment delivery component 305 are treatment delivery packages 300, as shown in FIG. 9.

The top layer of the treatment delivery component 305 is the support layer 320. The support layer 320 provides structure to the treatment delivery component 305 and a mounting surface for the removable coupling mechanism of the heater bottom cover 250 of the heater component 200. The support layer 320 includes an adhesive portion 322 at the outer edge for removably attaching the cutaneous treatment apparatus 100 to the cutaneous region for a treatment session. The adhesive portion 322 is located along the outer edge of the support layer 320 such that it extends beyond the outer boundary of the treatment portion 350 and directly contacts the cutaneous region for attachment. In some embodiments, the support layer 320 is flexible in order to allow the treatment delivery component 305 to conform to the various shapes on different cutaneous regions for maximum surface contact.

In the illustrated embodiment, the thermal distribution layer 330 is located on the bottom of the support layer 320. The thermal distribution layer 330 is a material with high thermal conductivity.

In an embodiment, the thermal distribution layer 330 comprises one or more thermally conductive materials. Non-limiting examples of thermally conductive materials include aluminum, silver, gold, copper, silicon, carbon, graphene, and the like. Further non-limiting examples of thermally conductive materials include metal nanoparticles, metal foils, metal alloys, metallic nanostructures, and the like. Further non-limiting examples of thermally conductive materials include silicon, carbon loaded resins, carbon black filled conductive epoxy resins, thermally conductive epoxies, thermally conductive plastics, and the like. In an embodiment, the thermal distribution layer 330 comprises a thermal grease composition. In an embodiment, the thermal distribution layer 330 comprises a polymerizable liquid matrix having large volume fractions of an electrically insulating, thermally conductive, filler. Non-limiting examples of thermally conductive fillers include aluminum oxide, boron nitride, zinc oxide, aluminum nitride, and the like. Further non-limiting examples of thermally conductive fillers include carbon black, carbon fibers, ceramic, graphite, metal particles, and the like. In an embodiment, the thermal distribution layer 330 comprises one or more thermally conductive polymeric composites. In an embodiment, the thermal distribution layer 330 comprises one or more thermally conductive epoxy adhesives filled with metal particles (e.g., silver particles, gold particles, copper particles, and the like).

Further non-limiting examples of thermally conductive materials include aluminum, aluminum nitride, aluminum nitride, aluminum oxide, aluminum oxide, ceramics, copper, cubic boron nitride, diamond, gold, hexagonal boron nitride, silicon carbide, silver, zinc oxide, and the like, and combinations thereof.

The thermal distribution layer 330 ensures that portions of the treatment delivery component 305 that are outside of the heater component footprint also receive heat during a treatment session. During use, heat is transferred from the heater component 200 through the thermal distribution layer 330 and spread throughout the treatment delivery component footprint. In some embodiments, the thermal distribution layer 330 is shaped such that the entire surface of the treatment portion 350 receives heat from the heater component 200. In other embodiments, the thermal distribution layer 330 is shaped to target heat to certain areas of the treatment portion 350.

In an embodiment, the thermal distribution layer 330 provides a substantially even temperature about a target temperature. In an embodiment, the thermal distribution layer 330 provides a substantially even temperature of about 42° Celsius+/−3° Celsius.

In an embodiment, the thermal distribution layer 330 provides a substantially even temperature of about less than 45° Celsius for a target duration. In an embodiment, the thermal distribution layer 330 provides a substantially even temperature of about 42° Celsius. In an embodiment, the thermal distribution layer 330 provides a substantially even temperature of about 42° Celsius+/−3° Celsius. In some embodiments, the thermal distribution layer 330 provides a substantially even temperature (i.e., +/−3° Celsius) across the areas of the treatment delivery component 305 inside and outside of the heater component footprint. However, in other embodiments, the thermal distribution layer 330 is designed to target more heat to specific areas of the treatment delivery component 305 as required for the desired effect, cutaneous region, and anticipated outcome of the treatment session. As shown in the illustrated embodiment, the thermal distribution layer 330 is used when the heater component footprint is smaller than the treatment delivery component footprint, because it allows for a wide range of shapes, sizes, and contours, without the need to change the shape of the heater component 200. However, in other embodiments, where the heater component footprint is the same size or larger than the treatment delivery component footprint, the thermal distribution portion is omitted (e.g., to reduce cost and manufacturing complexity).

Still referring to FIG. 9, the next layer of the treatment delivery component 305, below the thermal distribution layer 330, is the barrier layer 340. The barrier layer 340 is configured to prevent loss of the treatment formulation 352 of the treatment portion 350 (see FIG. 10). The barrier layer 340 ensures that the treatment formulation 352 is directed toward the cutaneous region as opposed to being absorbed by other components of the cutaneous treatment apparatus 100 or evaporated into the surrounding air. Although shown as a separate layer in FIG. 9, in some embodiments, the barrier layer 340 is not a separate layer, but is formed on the surface of another layer, for example, the thermal distribution layer 330 or the treatment portion 350.

The final layer of the treatment delivery component 305 is the treatment portion 350. The treatment portion 350 is configured to directly contact the cutaneous region during a treatment session. The treatment portion 350 is held in contact with the cutaneous region by the adhesive portion 322 of the support layer 320. Referring now to FIG. 10, the treatment portion 350 comprises the treatment formulation 352. As a non-limiting example, the treatment formulation 352 comprises a hydrogel containing at least one therapeutic compound. In some embodiments, the therapeutic compound contained in the treatment formulation 352 comprises a variety of cutaneous treatment formulations, including cosmetic and drug. In some embodiments of the present disclosure, each treatment portion 350 contains a single therapeutic compound. In other embodiments, multiple therapeutic compounds are combined within a single treatment portion 350 in order to apply multiple treatments during a single treatment session. As non-limiting examples, cutaneous treatments include: skin whitening, wrinkle reduction, skin toning, burn soothing, age spot removal, psoriasis control, vitamin infusion, eczema treatment, general moisturizing, cellulite smoothing, rash reduction, skin tightening, pain relief, and nicotine, testosterone, and birth control medication delivery.

During the treatment session, the heater component 200 transfers heat through the layers of the treatment delivery component 305 such that the heat reaches the treatment portion 350 and causes the therapeutic compound contained in the treatment formulation 352 to diffuse into the cutaneous region. In some embodiments, the therapeutic compound is “heat activated,” such that the application of heat increases diffusion from the hydrogel, cutaneous absorption, and/or efficacy of the compound.

In some embodiments of the present disclosure, the heat transferred to the treatment delivery component 305 does not reach the cutaneous region, but is contained within the cutaneous treatment apparatus 100. In other embodiments, the heat from the heater component 200 is transferred to the cutaneous region such that the cutaneous surface is heated to a desired temperature to promote percutaneous absorption of the therapeutic compound. In certain instances, cutaneous heating promotes percutaneous absorption of the therapeutic compound through the process of thermophoresis, where absorption is increased in response to the force of a temperature gradient. The enhanced percutaneous absorption of the therapeutic compound has advantages, including quicker delivery to the cutaneous region, further penetration into the cutaneous layers, and lower dosages of the therapeutic compound required in the treatment formulation 352. For example, the cutaneous temperature during a treatment session is between about 35° Celsius and 45° Celsius. During some treatment sessions, the temperature of the cutaneous region is in the range of about 42° Celsius to 44° Celsius. In some embodiments, heating the cutaneous region to a specified temperature, as described above, requires a higher output temperature at the heater component 200 due to the thermal resistance and losses within the various layers of the treatment delivery component 305.

Referring to FIGS. 11-16, mechanisms for removably coupling the heater component 200 to the treatment delivery component 305 are shown. The mechanisms shown in FIGS. 11-16 are not an exhaustive list of mechanisms, and any other suitable removable coupling mechanisms are also within the scope of the present disclosure.

Referring to FIGS. 11A and 11B, a “snap and catch” mechanism is shown. The mechanism includes at least one snap and catch attachment post 252 disposed on the lower surface of the heater bottom cover 250. A corresponding snap and catch receptacle 362 is formed within the support layer 320 of the treatment delivery component 305, as shown. To removably couple the heater component 200 to the treatment delivery component 305, each snap and catch attachment post 252 is aligned with a corresponding snap and catch receptacle 362 and inserted until there is an interlocking of components as shown in FIG. 11B. To remove the heater component 200 after the treatment session, the components are pulled apart such that each snap and catch attachment post 252 deforms to release the interlocking of the components.

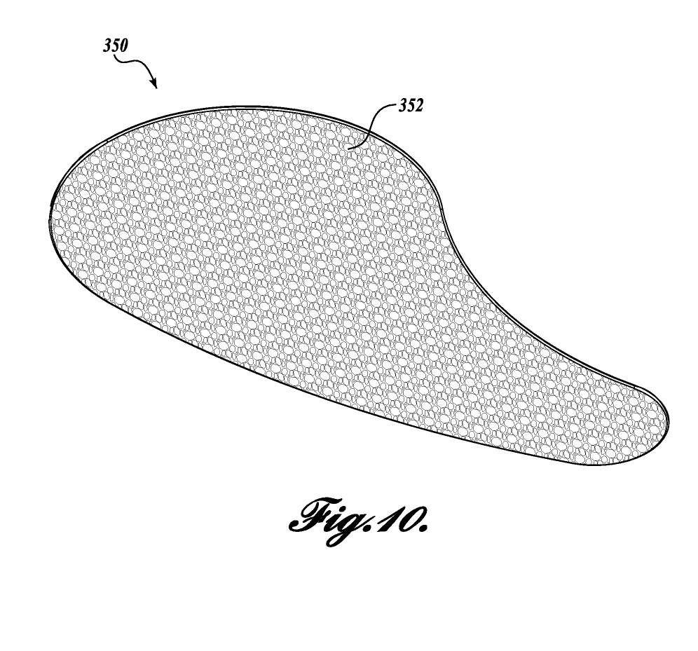

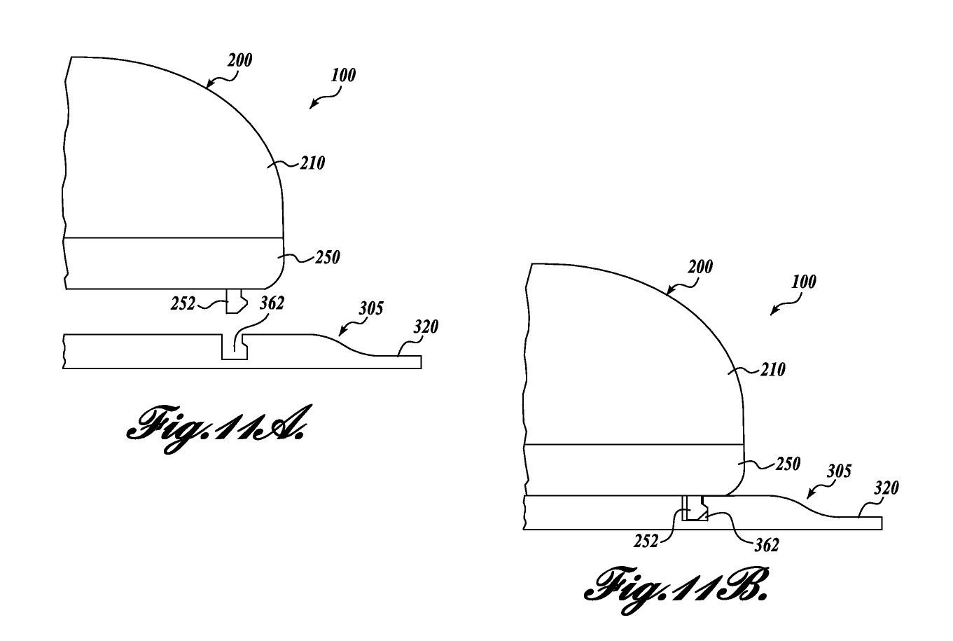

Referring to FIGS. 12A-12C, a “slot” mechanism is shown. The mechanism includes at least one linear rail 254 disposed on the lower surface of the heater bottom cover 250. A corresponding slot rail receptacle 364 is formed within the support layer 320 of the treatment delivery component 305, as shown. To removably couple the heater component 200 to the treatment delivery component 305, the end of each linear rail 254 is aligned with the end of each corresponding slot rail receptacle 362 and inserted until each linear rail 254 is fully engaged with the slot rail receptacle 364 as shown in FIG. 12C. To remove the heater component 200 after the treatment session, the components are slid apart such that each linear rail 254 is fully released from the slot rail receptacle 364.

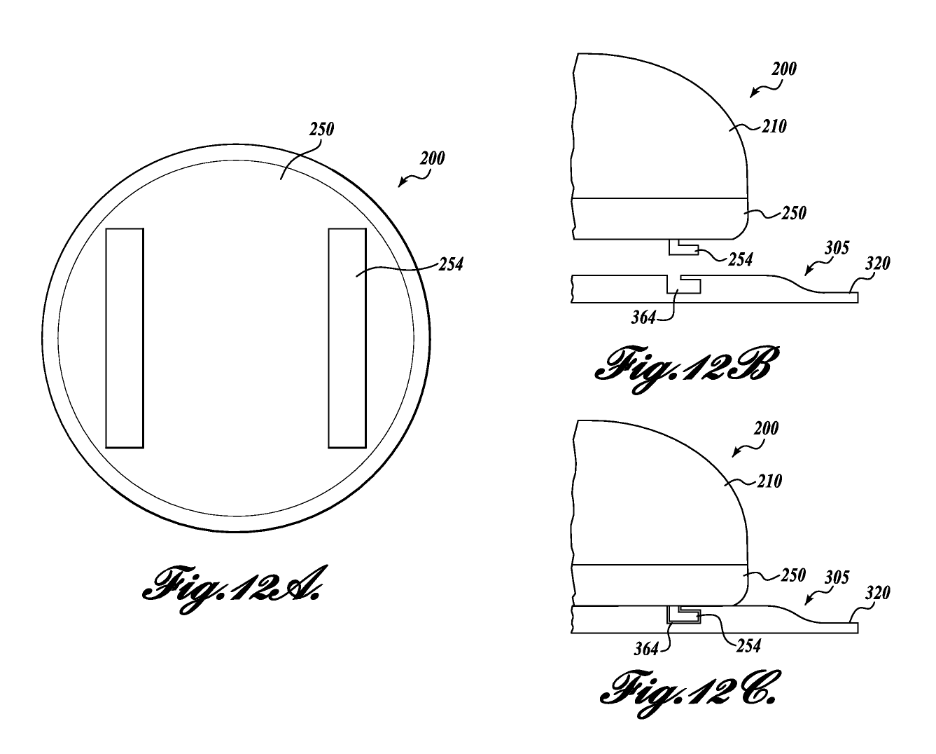

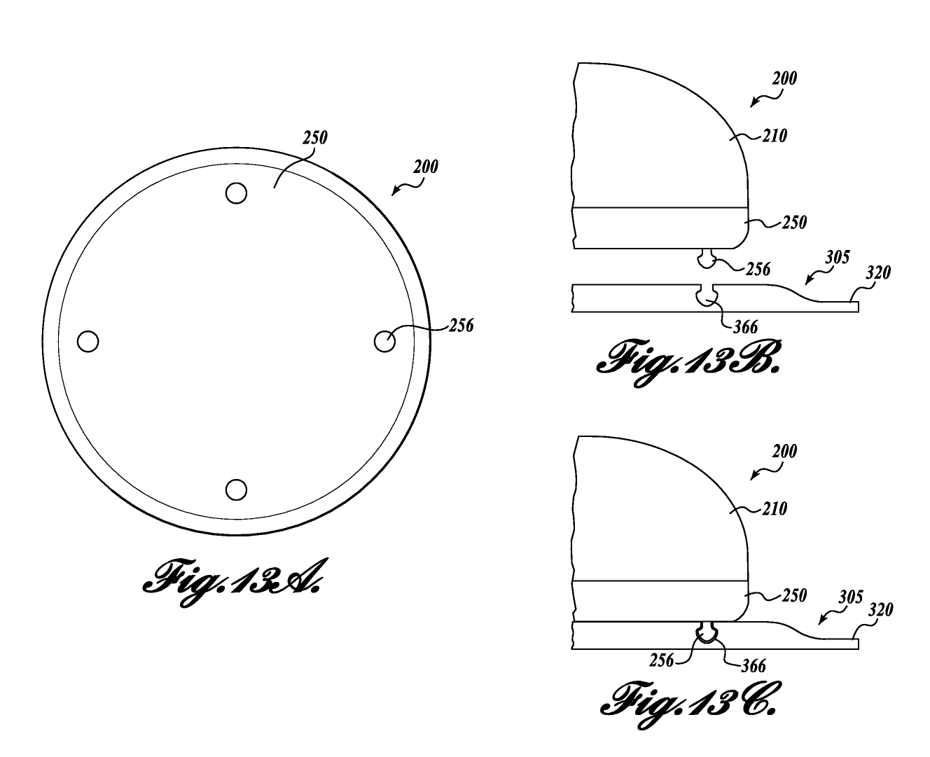

Referring to FIGS. 13A-13C, a “pin” mechanism is shown. The mechanism includes at least one pin attachment post 256 disposed on the lower surface of the heater bottom cover 250. A corresponding pin pocket receptacle 366 is formed within the support layer 320 of the treatment delivery component 305, as shown. To removably couple the heater component 200 to the treatment delivery component 305, each pin attachment post 256 is aligned with the corresponding pin pocket receptacle 366 and inserted until there is an interlocking of components as shown in FIG. 13C. To remove the heater component 200 after the treatment session, the components are pulled apart such that each pin attachment post 256 and each pin pocket receptacle 366 deform to release the interlocking of the components.

Referring to FIG. 14, a “magnetic” mechanism is shown. The mechanism includes at least one upper magnet 258 disposed on the lower surface of the heater bottom cover 250. A corresponding lower magnet 368 is disposed on the support layer 320 of the treatment delivery component 305, as shown. To removably couple the heater component 200 to the treatment delivery component 305, each upper magnet 258 is aligned with the corresponding lower magnet 368 and placed in contact such that the resulting magnetic force between the components provides the removable coupling mechanism. To remove the heater component 200 after the treatment session, the components are pulled apart such that each upper magnet 258 and each lower magnet 368 reach a distance apart such that the magnetic force dissipates.

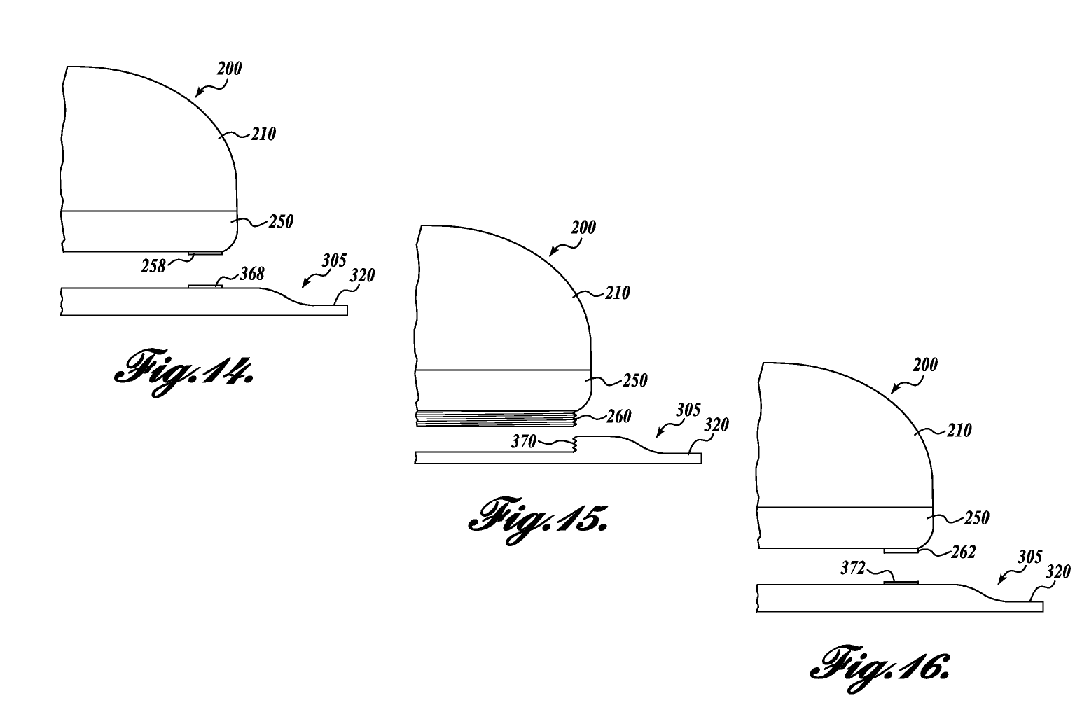

Referring to FIG. 15, a “threaded ring” mechanism is shown. The mechanism includes an upper threaded ring 260 disposed on the lower surface of the heater bottom cover 250. A corresponding lower threaded ring receptacle 370 is formed within the support layer 320 of the treatment delivery component 305, as shown. To removably couple the heater component 200 to the treatment delivery component 305, the upper threaded ring 260 is axially aligned with the corresponding lower threaded ring receptacle 370 and rotated until the thread ends are aligned. The heater component 200 is then twisted in a first direction (e.g., clockwise) such that the threads of the upper threaded ring 260 are engaged with the threads of the lower threaded ring receptacle 370. To remove the heater component 200 after the treatment session, the heater component 200 is rotated in a reverse direction (e.g., counter-clockwise) until the threads of the upper threaded ring 260 are disengaged from the threads of the lower threaded ring receptacle 370.

Referring to FIG. 16, the “adhesive” mechanism of FIGS. 2 and 3 is shown in greater detail. The mechanism includes at least one upper adhesive section 262 disposed on the lower surface of the heater bottom cover 250. Each corresponding lower adhesive section 372 is disposed on the support layer 320 of the treatment delivery component 305, as shown. To removably couple the heater component 200 to the treatment delivery component 305, each upper adhesive section 262 is aligned with a corresponding lower adhesive section 372 and placed in contact such that the adhesive combines to provide the removable coupling mechanism. To remove the heater component 200 after the treatment session, the components are pulled apart such that each upper adhesive section 262 and each lower adhesive section 372 release the adhesive bond.

In another aspect, methods of applying a treatment to a cutaneous region, using a cutaneous treatment apparatus as disclosed herein are provided. In one embodiment, the method includes: contacting the treatment surface to a portion of the cutaneous region, and heating the treatment portion to a treatment temperature with the heater. The heater component 200 provides heat to the treatment portion 350 to reach the treatment temperature. Heating of the treatment portion releases the therapeutic compound through the treatment surface towards the cutaneous region. In one embodiment, the method of applying the treatment includes attaching the cutaneous treatment apparatus to a cutaneous surface with an adhesive portion such that the treatment formulation is applied. In some embodiments, the treatment temperature is between about 35° Celsius and 45° Celsius. In other embodiments, a portion of the cutaneous region is heated with the heater.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

The embodiments of the invention in which an exclusive property or privilege is claimed are defined as follows:

1. A cutaneous treatment apparatus, comprising:
a heater component including a heater, the heater component having a heater component footprint defined by a heater component perimeter; and
a first treatment delivery component, including a treatment portion having a treatment portion footprint defined by a treatment portion perimeter;
the treatment portion including a treatment formulation including a therapeutic compound and a treatment surface configured to contact a cutaneous region;
wherein the heater component is removably couplable with the first treatment delivery component and configured to transfer heat to the treatment portion, and wherein a therapeutic compound is configured to release from the treatment portion through the treatment surface towards the cutaneous region upon heating.

2. The cutaneous treatment apparatus of claim 1, wherein the first treatment delivery component further includes a thermal distribution portion intermediate the treatment portion and the heater component, and wherein the thermal distribution portion is configured to distribute the heat to areas of the treatment portion outside the heater component footprint.

3. The cutaneous treatment apparatus of claim 1, wherein the heater component is further configured to transfer heat through the treatment surface towards the cutaneous region.

4. The cutaneous treatment apparatus of claim 1, wherein the first treatment delivery component further includes an adhesive portion for removably attaching the treatment delivery component to the cutaneous region, such that the treatment surface contacts the cutaneous region.

5. The cutaneous treatment apparatus of claim 4, wherein the adhesive portion expands beyond the treatment portion footprint to allow for contact with the cutaneous region.

6. The cutaneous treatment apparatus of claim 1, wherein the treatment portion footprint is at least 10% larger than the heater component footprint.

7. The cutaneous treatment apparatus of claim 1, wherein the treatment portion footprint is smaller than the heater component footprint.

8. The cutaneous treatment apparatus of claim 1, wherein the treatment formulation is a hydrogel.

9. The cutaneous treatment apparatus of claim 1, wherein the therapeutic compound is a cosmetic composition including one or more moisturizers, humectants, whiteners, brighteners, epidermal fillers, peptides, saccharides, or acids.

10. The cutaneous treatment apparatus of claim 1, wherein the therapeutic compound is a drug selected from the group consisting of hormone therapies, pain management, and smoking cessation.

11. The cutaneous treatment apparatus of claim 1, wherein the heater component is configured to removably couple with a second treatment delivery component after removal from the first treatment delivery component.

12. The cutaneous treatment apparatus of claim 1, wherein the treatment delivery component further includes a barrier intermediate the treatment portion and the heater component, wherein the barrier prevents diffusion of the therapeutic compound away from the treatment surface.

13. The cutaneous treatment apparatus of claim 1, wherein the heater is an electric heater.

14. The cutaneous treatment apparatus of claim 13, wherein the electric heater includes a ceramic insulator.

15. The cutaneous treatment apparatus of claim 1, wherein the heater component comprises a controller configured to control a temperature and a target duration of heat production by the heater.

16. The cutaneous treatment apparatus of claim 15, wherein the controller is configured to shut off power to the heater after the target duration of heat production has ended.

17. The cutaneous treatment apparatus of claim 1, wherein the heater component is removably couplable by an attachment mechanism selected from the group consisting of mechanical, magnetic, and adhesive.

18. The cutaneous treatment apparatus of claim 1, wherein the heater is powered by a battery.

19. The cutaneous treatment apparatus of claim 18, wherein the battery is rechargeable.

20. A method of applying a treatment to a cutaneous region, using a cutaneous treatment apparatus of claim 1, the method comprising:
contacting the treatment surface to a portion of the cutaneous region; and
heating the treatment portion to a treatment temperature with the heater, thereby releasing the therapeutic compound through the treatment surface towards the cutaneous region.

21. The method of claim 20, wherein heating the treatment portion to the treatment temperature with the heater includes heating the treatment portion between about 35° and 45° Celsius.

22. The method of claim 20, further comprising:
removably attaching a treatment delivery component to a cutaneous region with an adhesive portion.

23. The method of claim 20, wherein heating the treatment portion to the treatment temperature with the heater includes heating a portion of the cutaneous region.
